(19) United States
(12) Patent Application Publication
(10) Pub. No.: US 2023/0134587 A1
(43) Pub. Date: May 4, 2023

(54) BIOMARKER TESTING AND DIAGNOSTICS

(71) Applicant: University of Washington, Seattle, WA (US)

(72) Inventors: Sarah J. Iribarren, Seattle, WA (US); Barry R. Lutz, Seattle, WA (US); Daniel Leon, Seattle, WA (US); Marcus Rhodehamel, Seattle, WA (US); Jesse Keyes, Seattle, WA (US); Hannah Erdy, Seattle, WA (US); Mohamed Adil, Seattle, WA (US); Rishika Das, Seattle, WA (US); Kyle Goodwin, Seattle, WA (US)

(21) Appl. No.: 17/888,335

(22) Filed: Aug. 15, 2022

Related U.S. Application Data
(60) Provisional application No. 63/273,315, filed on Oct. 29, 2021.

(51) Int. Cl. G01N 33/50; G01N 33/493; G16H 20/10; G01N 21/01

(52) U.S. Cl. CPC G01N 33/5038; G01N 33/493; G16H 20/10; G01N 21/01; G01N 2021/0118; G01N 2033/4975

(57) ABSTRACT

Embodiments disclosed herein relate to apparatuses, systems, and methods for detecting biomarkers in biological samples present in assay tests using a control strip and indicator strip, where the control strip includes reagents that form a reactive gas which reacts with the biomarker on the indicator strip, if present. Methods of using the test apparatuses include taking a sending photographs of a used test apparatus to a medical provider to demonstrate adherence to a pharmaceutical treatment regimen.

### CROSS-REFERENCE TO RELATED APPLICATIONS

This application claims priority to U.S. Patent Application No. 63/273,315 filed on 29 Oct. 2021, the disclosure of which is incorporated herein, in its entirety, by this reference.

### STATEMENT REGARDING FEDERALLY SPONSORED RESEARCH

This invention was made with government support under Grant Numbers K23 NR017210 and R01 AI147129, awarded by the U.S. National Institutes of Health. The U.S. government has certain rights in the invention.

### BACKGROUND

Lateral flow assay tests can be used to detect analytes such as antibodies or chemicals present in biological samples. Lateral flow assay tests allow users to monitor health conditions in locations other than medical facilities.

Globally, about a quarter of the population, or 1.7 billion people, are infected with latent or asymptomatic tuberculosis (“TB”) infection and have a roughly 10 percent chance of developing the active disease. The World Health Organization's End TB Strategy milestone to reduce the incidence of TB by 20% during 2015-2020 has not been met, with only a 6.3% reduction rate in 2019. The End TB Strategy envisions a TB-free world by 2035, with zero deaths, zero new cases and no suffering due to TB.

### SUMMARY

Embodiments disclosed herein relate to apparatuses, systems, and methods for detecting biomarkers in biological samples present in assay tests using a control strip and indicator strip spaced from the control strip by a distance, where the control strip includes reagents that form a reactive gas which reacts with the biomarker, if present, on the indicator strip to form a colored compound.

In an embodiment, a test apparatus for detecting a drug metabolite is disclosed. The test apparatus includes a control strip including at least three reagents formulated to make a reactive gas upon mixing. The test apparatus includes an indicator strip spaced from the control strip by a distance, the indicator strip including a color change reagent formulated to form a colored compound upon reaction with a biomarker and the reactive gas.

In an embodiment, a method for testing for a drug metabolite is disclosed. The method includes applying a biological sample onto a test apparatus for a biomarker, the test apparatus including a control strip including at least three reagents formulated to make a reactive gas upon mixing, and an indicator strip spaced from the control strip, the indicator strip including a color change reagent formulated to form a colored compound upon reaction with a biomarker and the reactive gas. The method includes allowing the biological sample to travel through the control strip and indicator strip to a terminal region thereof for a duration of time sufficient to allow the color change reagent to form the colored compound if the biomarker is present in the biological sample. The method includes identifying a presence or absence of the colored compound on the indicator strip.

In an embodiment, a method for treating tuberculosis is disclosed. The method includes receiving an electronic copy of a photograph of a completed biomarker test via a wireless network, the completed biomarker test including a test apparatus having, a control strip including at least three reagents formulated to make a reactive gas upon mixing, and an indicator strip spaced from the control strip, the indicator strip including a color change reagent formulated to form a colored compound upon reaction with a biomarker and the reactive gas. The method includes identifying a presence or absence of the colored compound on the indicator strip. The method includes providing a treatment regimen responsive to the presence or absence of the colored compound on the indicator strip.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is an isometric view of a test apparatus for detecting a biomarker, according to an embodiment.

FIG. 2 is an exploded isometric view of the test apparatus of FIG. 1.

FIG. 3 is an isometric view of a control strip and an indicator strip, according to an embodiment

FIG. 4 is an isometric view of a base, according to an embodiment.

FIG. 5A is an exploded isometric view of a test apparatus, according to an embodiment.

FIG. 5B is an isometric view of the test apparatus of FIG. 5A, according to an embodiment.

FIG. 6 is a flow diagram of a method for testing for a biomarker and treating a patient for a condition, according to an embodiment.

### DETAILED DESCRIPTION

Embodiments disclosed herein relate to test apparatus(es) for detecting biomarkers such as metabolites of a pharmaceutical composition, systems for reading the test apparatus(es), and methods of using and reading the test apparatus(es). The test apparatuses include an indicator strip and a control strip spaced from the indicator strip by a distance. The control strip includes a plurality of reagents formulated to make a reactive gas upon mixing and the indicator strip includes a color change reagent formulated to form a colored compound upon reaction with the reactive gas and a biomarker (e.g., metabolite) of a pharmaceutical composition. By separating the indicator strip from the control strip while maintaining the strips in proximity to each other, the test apparatuses herein help prevent the solid and liquid reagents on the respective strips from cross-contaminating each other to provide false-positives, but allow the reactive gas formed from the control strip to react with the color change reagent and any biomarker on the indicator strip to form a colored compound. The colored compound visually indicates that the biomarker is present in a biological sample on the indicator strip.

The test apparatuses disclosed herein can be used by a user (e.g., patient), photographed, and the photograph electronically sent to a medical care professional to demonstrate that a patient is or is not complying with a treatment (e.g., pharmaceutical treatment regimen). The test apparatuses, systems, and methods disclosed herein may be used for monitoring compliance with treatment regimens for a myriad of health conditions. For example, the test apparatuses, systems, and methods herein are particularly useful for monitoring compliance with pharmaceutical treatment for TB. Despite being largely curable, in 2018 TB killed an estimated 1.5 million people (about 4,100 deaths per day) with 10 million new cases of the disease worldwide, most of which live in low- and middle-income countries. The test apparatuses, systems, and methods disclosed herein are particularly useful in areas where medical services are not readily available, such as rural areas. In rural or poorer areas, medical professionals and facilities may not be present or readily accessible, which makes access to traditional in-person medical care and treatment monitoring impossible or impractical for many people. However, nearly 95% of the world's population lives in an area that has cellular phone coverage. Accordingly, using cellular and wireless network communications to demonstrate treatment compliance or adherence with the test apparatuses through electronic photographs of completed tests disclosed herein provides a meaningful solution for medical professionals and global health outcomes.

Treatment non-adherence is complex, costly, and can be a major obstacle to TB control. Treatment non-adherence can reduce cure rates, leads to more severe disease, prolongs infectiousness, and contributes to the emergence of multi-drug resistant TB strains. Drug resistant TB is more contagious, costly, and deadly. Accordingly, the prevention of the spread of the disease and development of resistance is important to global health. TB patients face multiple barriers to treatment adherence, including a long course of treatment, social stigma, fear, discrimination, poverty, poor pre-existing physical and mental health, poor clinical understanding of the disease and its treatment, high depression prevalence, medication side effects, local availability of healthcare, and difficulties accessing care. Innovative ways to monitor and support treatment adherence are described herein to mitigate adverse disease outcomes and the rapid emergence of drug resistance. In combination with the test apparatuses disclosed herein, mobile health (mHealth) tools can empower patients to take a more active role in managing their health while supporting healthcare teams to identify patients most at risk for stopping treatment and provide timely, individualized support, remotely.

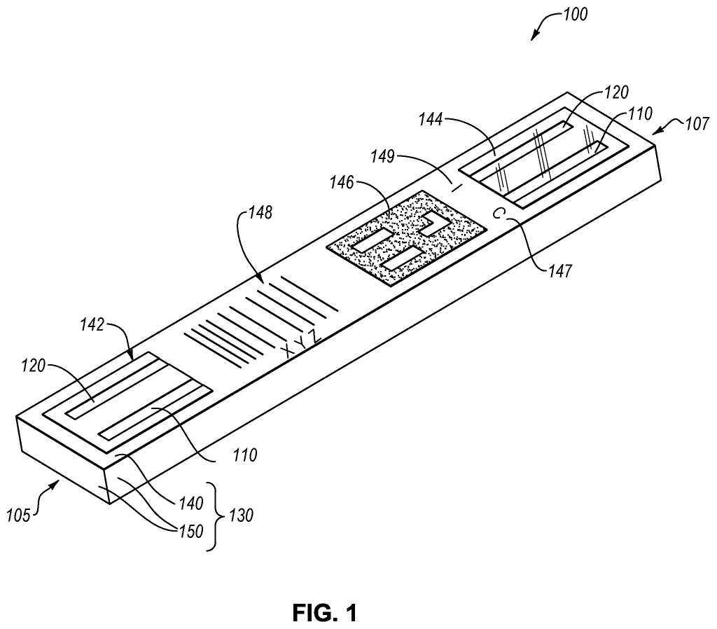

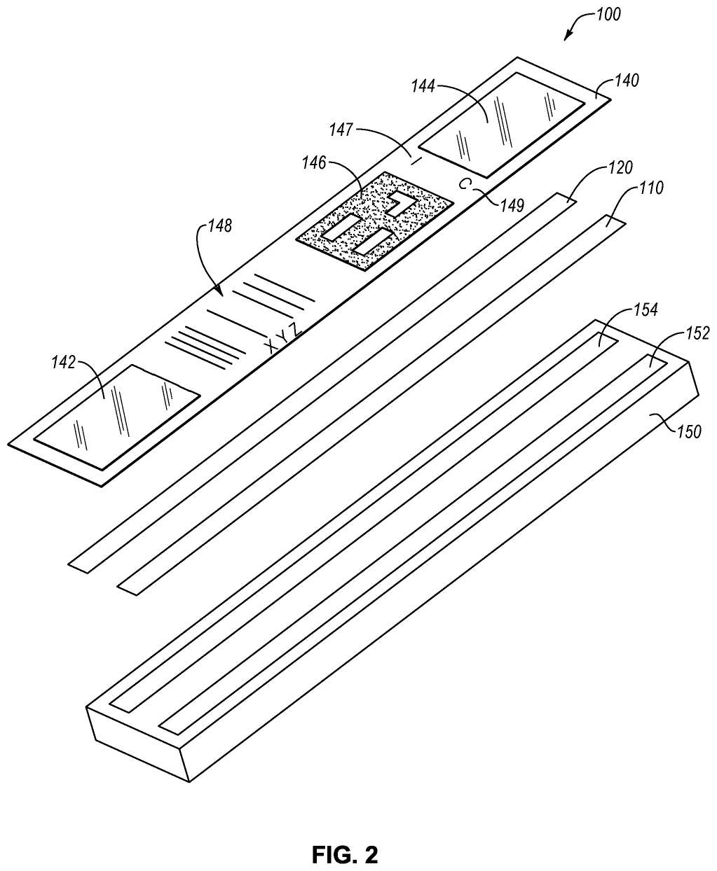

FIG. 1 is an isometric view of a test apparatus 100 for detecting a biomarker, according to an embodiment. FIG. 2 is an exploded isometric view of the test apparatus 100 of FIG. 1, according to an embodiment. The test apparatus 100 is an assay test for a biomarker of a drug, such as a drug metabolite. The test apparatus 100 includes a control strip 110 and an indicator strip 120 spaced from the control strip 110 by a distance. The test apparatus 100 may include packaging 130 containing the control strip 110 and the indicator strip 120. The control strip 110 includes a plurality of, such as at least three, reagents formulated to make a reactive gas upon mixing. The indicator strip 120 includes a color change reagent formulated to form a colored compound upon reaction with a biomarker and the reactive gas. The packaging 130 includes a base 150 and a cover 140 sealing (from the external environment) at least a portion of the control strip 110 and the indicator strip 120 therebetween.

The control strip 110 is configured as an assay strip. The control strip 110 includes material selected to move the biological sample (e.g., liquid) therethrough, such as via capillary action, osmotic pressure, wicking, gravity, or the like. The control strip 110 may include an elongated body of one or more of paper, fabric, foam, wicking material, or the like. The material of one or more portions of the control strip 110 may include one or both of natural (e.g., cotton, paper) or synthetic materials (e.g., glass fibers, polymer fibers). One or more portions of the indicator strip 120 may include a hydrophobic coating thereon. The porosity of the material of the control strip 110 may be selected to move the biological sample therethrough at a selected rate. The control strip 110 includes a plurality of reagents, such as at least three reagents, thereon. The plurality of reagents formulated to make a reactive gas upon mixing (e.g., reacting). The plurality of reagents may be disposed longitudinally along the control strip 110 to cause the reaction therebetween to occur at a selected speed and to produce reactive gas(es), as a product, at a selected position therealong. For example, the reagents may be positioned on the control strip 110 in positions such that the reactive gas is produced at a terminal region 107 of the control strip 110 and test apparatus 100.

The plurality of reagents, such as at least three reagents, may be selected to form a reactive gas upon mixing. In such examples, the plurality of reagents may include reagents selected to form a reactive gas that will react with a color change reagent and biomarker on the indicator strip 120 to form a colored compound on the indicator strip 120 that is a color that was not initially present on the indicator strip. Biological markers or biomarkers can refer to a measurable indicator of some biological state or condition and may include metabolite(s), drug molecule(s), or the like. Metabolite can refer to an intermediate or end product of metabolism. Metabolites have various functions, such as fuel, structure, signaling, stimulatory and inhibitory effects on enzymes, catalytic activity of their own, defense, and interactions with other organisms.

The plurality of reagents may be any reagents selected to react with a biomarker of interest, such as a biomarker for a TB drug metabolite. For example, the at least three reagents may include sodium chloro(4-methylbenzene-1-sulfonyl)azanide (“Chloramine T”), 2-hydroxypropane-1,2,3-tricarboxylic acid (“citric acid”), and potassium thiocyanate (“KSCN”). In such examples, the reactive gas is carbononitridic chloride (“cyanogen chloride”). Cyanogen chloride is toxic to humans so the location of the reactive gas (e.g., terminal region of the control strip 110) should be sealed from the external environment to limit or prevent human contact. In such examples, the cyanogen chloride may react with a TB drug biomarker, such as an isoniazid drug or metabolite thereof (e.g., 4-pyridinecarboxylic acid hydrazide (“INH”), 4-pyridinecarboxylic acid (“INA”), or derivatives thereof) to form an intermediate. The intermediate may be formulated to react with a color change reagent, such as 1,3-diazinane-2,4,6-trione (“barbituric acid”). Accordingly, if the biomarker is present, the color change reagent and reactive gas will react with it to produce the colored compound and provide a visual indication that the biomarker is present in the biological sample. The at least three reagents, color change reagent, and biomarkers may include alternative chemicals than those listed above.

The distance of the spacing between the control strip 110 and the indicator strip 120 may be selected to allow the reactive gas to contact the indicator strip 120 without allowing solid or liquid components on or from the control strip 110 to contact the indicator strip 120. Accordingly, the reactive gas may be used to react to form the colored compound on the indicator strip 120 without contamination from any other components on the control strip 110.

The separated control strip 110 and indicator strip 120 allows the use of an additional color change reagent to be used on the control strip 110 to form an additional colored compound thereon. The additional colored compound provides a visual indication that the biological sample has travelled through the control strip 110 from the initial region to the terminal region thereof. In such examples, the additional color change reagent may be used in combination with the biomarker or a biomarker analogue disposed on the control strip 110. The biomarker or biomarker analogue may be similar or identical to the biomarker of interest for which the test is formulated, such as an isoniazid drug or metabolite thereof (e.g., INH, INA, AcINH, or derivatives thereof). The biomarker or biomarker analogue may include INA, INH, AcINH, niacin, or the like. In such examples, the additional color change reagent on the control strip 110 may be similar or identical to the color change reagent on the indicator strip 120. For example, one or both of the color change reagent or the additional color change reagent includes barbituric acid. In some examples, the additional color change reagent on the control strip 110 may differ from the color change reagent on the indicator strip 120 in one or more of chemical species or concentration, such as to provide a different colored compound (e.g., provide a different color) or a more intense color upon reaction with the biomarker and reactive gas. In some examples, 1,3-dimethylbutylamine (“1,3-DMBA”) may be used in place of or in addition to barbituric acid for one or more of the color change reagent or the additional color change reagent. 1,3-DMBA may have greater solubility in the biological sample than barbituric acid and therefore improve availability for reaction with the intermediate and help reduce the amount of color change reagent needed for production.

The amount of biomarker or biomarker analogue on the control strip 110 may be present in an amount selected to be greater than the amount sufficient to react with all of the reactive gas produced, such as two times, three times, five times, 10 times, or 1000 times greater. The amount of biomarker or biomarker analogue may be less than two times greater than the amount of reactive gas, such as 1%, 2%, 3%, 5%, 10%, 25%, 50%, 75%, 85%, or 95% more than the reactive gas. As little as a 0.20 μmol excess biomarker or biomarker analogue to the reactive gas may be used. Such amount(s) may be in the terminal region of the control strip 110, such as at the end of the control strip 110. Such embodiments allow the reactive gas to form on the control strip 110, spread to the indicator strip 120 to react with components thereon, and be substantially completely consumed by the indicator strip 120 and/or control strip 110, to eliminate toxic substances produced in the test apparatus 100, after use. For example, the biomarker (e.g., INA) concentration in the control strip may be the saturation concentration (5.7 mg/mL) and by applying 14 passes of solution (184.8 μL) to the control strip, the reactive gas (e.g., cyanogen chloride) should be completely neutralized by the biomarker on the control strip. In embodiments, the volume of created reactive gas cyanogen chloride is relatively small (about 0.2 mL). The amount of gas is then reduced as some portion of it reacts with the biomarker (e.g., INH metabolite). In the instance in which the user is not taking the medication and the metabolite is not present in the urine, the maximum amount of reactive gas is present and not being used in the conversion of the biomarker into the colored compound (e.g., dye) on the indicator strip 120. In some examples, the maximum amount of reactive gas that should be produced from each control strip is 0.51 milligrams (mg) [204 μL at STP]. With the biomarker or biomarker analogue in the control strip 110, the reactive gas produced can be completely consumed by the biomarker or biomarker analogue therein. This amount is below the safe exposure limit Such embodiments allow for safe use of the test apparatuses herein even when there is not biomarker in the biological sample. It should be understood that the reactive gas reacts with both the biomarker on the indicator strip 120 and the biomarker or biomarker analogue on the control strip 110 when present.

The test apparatus 100 includes the indicator strip 120. The indicator strip 120 is configured as an assay strip. The indicator strip 120 includes material selected to move the biological sample (e.g., liquid) therethrough, such as via osmotic pressure, wicking, gravity, or the like. For example, the indicator strip 120 may include an elongated body of one or more of paper, fabric, foam, wicking material, or the like. The material of one or more portions of the indicator strip 120 may include one or more of natural (e.g., cotton, paper) or synthetic materials (e.g., glass fibers, polymer fibers). For example, the indicator strip 120 may include a main wicking material of grade 222 cotton fibers (e.g., AHLSTROM-MUNKSJÖ—Grade 222 Cotton blotting paper), and connective wicking material of glass fibers (e.g., EMD MILLIPORE—GFDX Glass Fiber), all on a backing card (e.g., vinyl backing card from DCN—MIBA-020). One or more portions of the indicator strip 120 may include a hydrophobic coating thereon. The porosity of the material of the indicator strip 120 may be selected to move the biological sample therethrough at a selected rate. The indicator strip 120 includes the color change reagent thereon, such as barbituric acid. The color change reagent is formulated to react with the reactive gas and the biomarker (if present in the biological sample) to form the color compound. Accordingly, the indicator strip 120 provides a visual indication that the biomarker is present in the biological sample. The color change reagent may be disposed at one or more points longitudinally along the indicator strip 120 to cause the reaction(s) therewith to occur at a selected position therealong. For example, the color change reagent may be positioned in a medial region or portion of the indicator strip 120 and test apparatus 100.

By limiting the presence of the plurality of reagents to the control strip 110, false positives may be avoided and the amount of toxic reactive gas produced in the test apparatus 100 may be kept as small as possible. In such examples, the reactive gas, such as cyanogen chloride may react with the a TB drug biomarker if present in the biological sample and with the color change reagent. If the biomarker is present, the color change reagent and reactive gas will react with it to produce the colored compound and provide a visual indication that the biomarker is present in the biological sample. In the case of barbituric acid, the colored compound is blue-purple. The amount of color change reagent on the indicator strip 120 may be selected to provide a selected amount of color compound (e.g., intensity of color) at the terminal region 107 of the indicator strip 120. For example, if a darker color is desired to make reading the test easier, then a greater amount of color change reagent may be used than if a lighter color is desired.

The distance of the spacing between the control strip 110 and the indicator strip 120 may be selected to allow the reactive gas to contact the indicator strip 120 without allowing solid or liquid components on or from the control strip 110 to contact the indicator strip 120. Accordingly, the reactive gas may be used to react to form the colored compound on the indicator strip 120 without contamination from any other components on the control strip 110.

The distance between the control strip 110 and the indicator strip 120 is selected to prevent components of the control strip 110 from contacting components of the indicator strip 120 yet still allow the reactive gas to reach and react with biomarker and color change reagent on the indicator strip 120. The distance between the respective strips maybe at least 1 mm apart, such as 1 mm to 2 cm, 3 mm to 7 mm, 5 mm to 1 cm, 7 mm to 1.5 cm, 1 cm to 2 cm, less than 2 cm, or less than 1 cm. In some embodiments, an additional material such as an inert spacer material may be positioned between the control strip 110 and the indicator strip 120.

In embodiments, the indicator strip 120 may be substantially parallel to the control strip 110 where the distance between the strips is substantially uniform along the length of the strips. Such a configuration is particularly useful because it provides an initial region and terminal region at the same point on the test apparatus 100 for both the indicator strip 120 and the control strip 110. The parallel configuration allows the biological sample (e.g., urine test) to travel through the indicator strip 120 and control strip 110 at substantially the same speed. In such examples, an additional colored compound on the control strip 110 indicates that the biological sample has also traveled to the terminal region of the indicator strip 120, verifying that the test is completed. If no colored compound is visible on the indicator strip 120, then it can be assumed that the biomarker is not present in the biological sample and the patient has therefore not been complying with a pharmaceutical treatment regimen, such as for TB. If both the colored compound and the additional colored compound are visible on the respective strips, then it can be assumed that the biomarker is present in the biological sample and the patient is complying with the pharmaceutical treatment regimen.

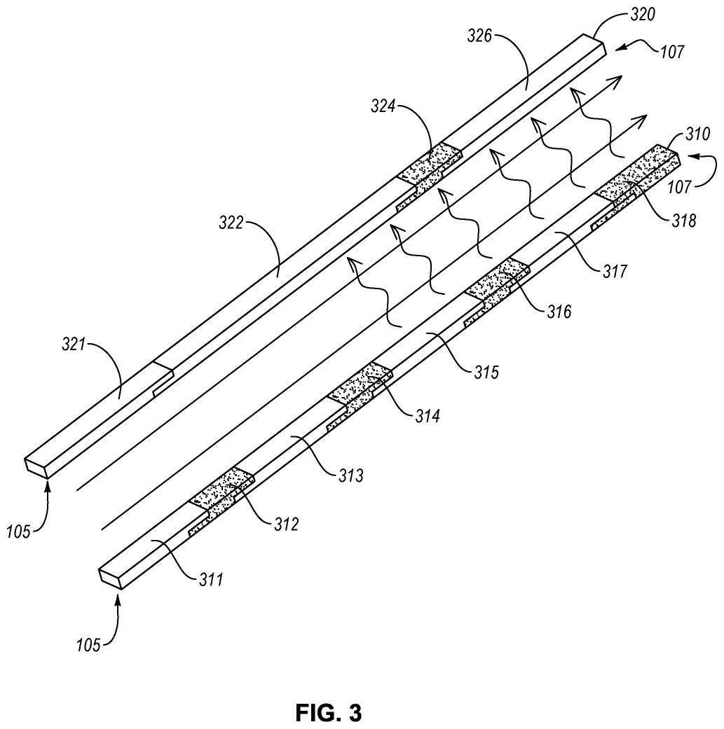

The position of the chemical components on the control strip 110 and indicator strip 120 may differ. FIG. 3 is an isometric view of a control strip 310 and an indicator strip 320, according to an embodiment. The control strip 310 may be similar or identical to the control strip 110 in on or more aspects and the indicator strip 320 may be similar or identical to the indicator strip 120 in one or more aspects. The control strip 310 may include the plurality of reagents thereon or therein such as each applied to a single piece of material at different longitudinal positions thereon or in different sections of material in contact with each other to form the control strip 310. FIG. 3 depicts the latter in an initial state prior to application of a biological sample thereto.

The control strip 310 may include a plurality of control sections 311-318, wherein at least some of the sections include some of the plurality of reagents (e.g., at least three reagents) thereon. For example, a first control section 311 may include no reagents thereon. The first control section 311 may be an initial portion with no reagent to receive the biological sample thereon. The second control section 312 may include potassium thiocyanate and citric acid thereon. The third control section 313 may include no reagent. The fourth control section 314 may include chloramine T thereon. The fifth control section 315 may include no reagent. The sixth control section 316 may include the color change reagent, such as barbituric acid, therein or thereon. The seventh control section 317 may include no reagent. The optional eighth control section 318 may include the biomarker or an analogue of the biomarker. The control sections 313, 315, or 317 of the control strip 310 are utilized to prevent cross-contamination of sections containing reagents prior to use and may be utilized to allow time for reactions to occur between reagents and reaction products on the control strip 310. In some embodiments, one or more of control sections 313, 315, or 317 of the control strip 310 may be omitted. While depicted as separate sections, the control sections 311-318 may be implemented on a single piece of material, such as with reagents longitudinally spaced from each other.

The various control sections 311-318 may be constructed of identical materials to move the biological sample through the control strip at a substantially continuous speed. The various control sections 311-318 may be constructed of different materials, such as to selectively control the speed of fluid flow therethrough by utilizing materials with different wicking or osmotic properties at different longitudinal points therein. In embodiments, the terminal control section 318 may include a material with the strongest wicking properties, to retain the biological sample and reagents therein.

As the biological sample travels from first control section 311 at the initial region 105 to the eighth or terminal control section 318 at the terminal region 107 of the control strip 310, various reactions take place to form the reactive gas, and in some embodiments, the additional colored compound. For example, in the instance of a test for an isoniazid drug biomarker, the biological sample (e.g., urine) is applied to the first control section 311 and travels through the control strip 310 toward the terminal region 107, via wicking, capillary action, osmotic pressure, gravity, or the like. Assuming the biomarker (INA, AcINH, or INH) for an isoniazid drug (INH) is present in the biological sample, the biomarker may be carried through the control strip 310 from the initial region 105 to the terminal region 107. The biomarker may be a reaction product of the isoniazid drug with one or more materials in the body of the patient, such as a reaction product of acetylation and hydrolysis of the isoniazid drug in metabolic reactions in the body of the patient. Such a reaction is shown as Reaction 1 below:

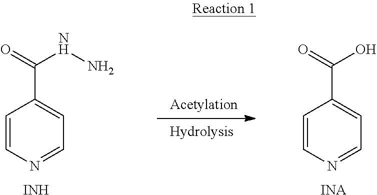

The biological sample, including the biomarker therein, travels from the first control section 311 to the second control section 312, where it mixes with citric acid and potassium thiocyanate therein. The biological sample, containing the biomarker, citric acid, and potassium thiocyanate travels through the third control section 313 which contains no additional reagent. In the fourth control section 314, the biological sample mixes with chloramine T therein to form the reactive gas cyanogen chloride. Such a reaction is shown as Reaction 2 below:

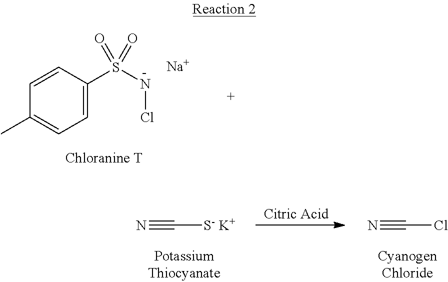

The chloramine T and potassium thiocyanate reactants in the biological sample continue to react to form the cyanogen chloride in the control sections 314-318 as the respective reactants are consumed. The reactive gas may spread from the control strip 310 to the indicator strip 320 to participate in reactions on the indicator strip 320. The cyanogen chloride reacts with the biomarker (e.g., INA) in the biological sample to form a reaction intermediate. The reactive gas breaks the pyridine ring of the biomarker to then form an intermediate and eventually the colored compound or dye that leads to visible color change. The reaction of cyanogen chloride with the drug metabolite INA is shown as Reaction 3 below:

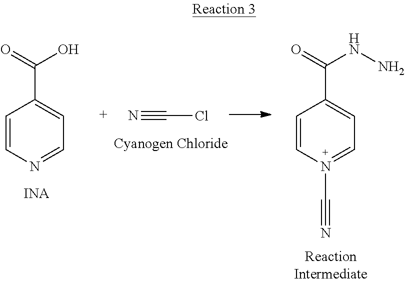

The reaction intermediate hydrolyzes from the water in the biological sample (e.g., water in urine) to form a glutaconic aldehyde intermediate. This reaction is shown as Reaction 4 below:

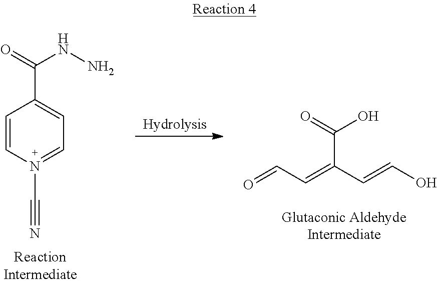

The biological sample, containing the biomarker, citric acid, potassium thiocyanate, cyanogen chloride, reaction intermediate, and glutaconic aldehyde intermediate travels through the fifth control section 315 which contains no additional reagent. In the sixth control section 316, the biological sample may mix with an additional color change reagent when present, such as barbituric acid. The glutaconic aldehyde in the biological sample reacts with the barbituric acid in an aldol condensation reaction to form the additional colored compound, a highly conjugated product with a blue-purple color. The reactions between cyanogen chloride, INA, and the derivatives thereof with barbituric acid occur at a pH of about 5 (e.g., 4.5 to 5.5). Accordingly, the control strip 310 may include one or more chemicals (e.g., buffers) thereon to control the pH of the biological sample to a selected amount at selected locations thereon, such as to about 5 pH. The barbituric acid and glutaconic aldehyde reaction is shown as Reaction 5 below:

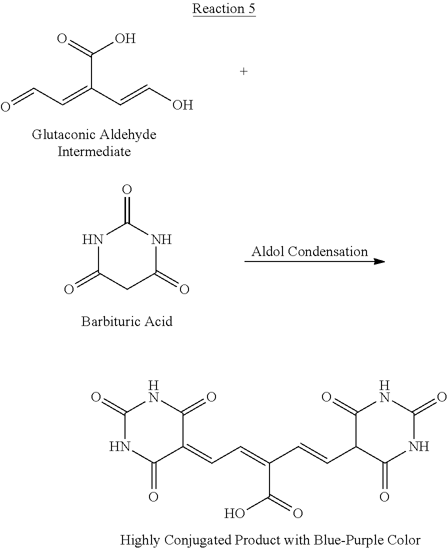 [SMILES: O=C/C=C(/C=C/O)C(=O)O ; O=C1CC(=O)NC(=O)N1 ; O=C1NC(=O)C(=C/C=C(/C=C/C2C(=O)NC(=O)NC2=O)C(=O)O)C(=O)N1]

Reaction 5 may progress in sixth control section 316 and seventh control section 317, which contains no additional reagent. Reaction 5 may take place in the eighth control section 318 as well.

As shown in FIG. 3, the eighth control section 318 may include, in an embodiment, the biomarker or a biomarker analogue to ensure that the control strip 310 has a color change indicating that the biological sample has flowed through the entire control strip 310 and reached the terminal region 107. For example, the biomarker or biomarker analogue in the eighth control section 318 ensures that the plurality of reagents and products thereof (e.g., cyanogen chloride) react with the additional color change reagent to form the additional colored compound. In such embodiments, the user of the test will know that the biological sample has reached the terminal region 107 and is therefore correctly used (e.g., any results—positive or false—are a result of the biomarker being present or not in the biological sample, and not because of a user or chemical error) and complete. If the indicator strip 320 does not have a colored compound thereon, it can be assumed that the biological sample does not contain the biomarker, and the patient has not complied with a pharmaceutical treatment regimen. Further, by including the biomarker or biomarker analogue on the control strip 310 (and 110) in a quantity sufficient to react with at least all of the reactive gas produced from the control strip and test, the biomarker or biomarker analogue acts as a reagent for consuming any excess reactive gas and neutralizing it. The eighth control section 318 may include a material with the greatest wicking properties to ensure that the biological sample is preferentially drawn and retained therein to make sure the additional colored compound, if present, is visible in the terminal region 107.

In some embodiments, a unique color change reagent may be located in the eighth control section 318 to indicate that the biological sample has reached the terminal region 107. The unique color change reagent may be formulated to change color upon contact with water or any of the other contents of the biological sample at the eighth control section 318.

While the reagents disclosed above are located in specific control sections, the reagents may be located in different control sections, such as locating the barbituric acid in the second control section 312 and moving each of the other reagents two control sections farther on the control strip 310, respectively.

The indicator strip 320 may include a plurality of indicator sections 321-326, wherein at least one of the indicator sections includes the color change reagent thereon. For example, a first indicator section 321 may include no reagents thereon. The first indicator section 321 may be an initial portion to receive the biological sample thereon. The second indicator section 322 may include no reagent, such as only comprising a wicking material. The third indicator section 324 may include the color change reagent, such as barbituric acid therein or thereon. The fourth indicator section 326 may include no reagents therein. In some embodiments, one or more of indicator sections 321 or 322 of the control strip 310 may be omitted. While depicted as separate sections, the indicator sections 321-326 may be implemented on a single piece of material.

The various indicator sections 321-326 may be constructed of identical materials to move the biological sample through the indicator strip at a substantially continuous speed. The various indicator sections 321-326 may be constructed of different materials, such as to selectively control the speed of fluid flow therethrough by utilizing materials with different capillary, wicking, or osmotic properties at different longitudinal points therein. In embodiments, the terminal or fourth indicator section 326 may include a material with the strongest wicking properties, to retain the biological sample, reagents, and reaction products in the terminal region 107.

As the biological sample travels from first indicator section 321 at the initial region 105 to the sixth or terminal indicator section 326 at the terminal region 107 of the indicator strip 320, various reactions take place between the biomarker (if present) the color change reagent and the reactive gas from the control strip 310. For example, the biological sample (e.g., urine) is applied to the first indicator section 321 and travels through the indicator strip 320 toward the terminal region 107, via wicking, capillary action, osmotic pressure, gravity, or the like. Assuming the biomarker (INA, INH, or AcINH) for an isoniazid drug (INH) is present in the biological sample, the biomarker may be carried through the indicator strip 320 from the initial region 105 to the terminal region 107. The biomarker may be the reaction product according Reaction 1 above.

The subsequent indicator sections 322-326 after the first indicator section 321 may have sequentially higher wicking, capillary, osmotic or other properties than the first indicator section 321 to preferentially move the biological sample therethrough. The second indicator section 322 may include no reagents therein. The third indicator section 324 may include the color change reagent therein, such as barbituric acid. As the reactive gas contacts the biomarker in the biological sample on the indicator strip 320, the biomarker therein reacts with the reactive gas and color change reagent to form a colored compound. For example, the cyanogen chloride from the control strip 310 reacts with the biomarker (ISA) on the indicator strip 320 according to Reaction 3 above and the reaction intermediate is formed and eventually reacts (e.g., hydrolyzes) to form a glutaconic aldehyde intermediate according to Reaction 4. As the glutaconic aldehyde derivative reacts with the barbituric acid in the third indicator section 324 and continues into the fourth indicator section 326, the colored compound (e.g., high conjugated produce having blue-purple color) is formed on the indicator strip 320 according to Reaction 5. The presence of the blue-purple color on the indicator strip 320 shows that the biomarker is present in the biological sample and that the patient has followed the pharmaceutical treatment regimen for the treatment of TB.

In some examples, one or both of the control strip 310 or the indicator strip 320 may include one or more mordanting materials thereon, such as in the terminal end regions thereof to localize and capture the colored compound therein. Such mordanting materials may include one or more of melamine, tannic acid, chitosan, or the like. Mordanting materials may be particularly useful for detecting low levels of biomarker (e.g., drug metabolite).

Alternative reagents and color change reagents may be used to indicate the presence of biomarkers other than the ISH or ISA described in the above example.

While an example testing apparatus and reagents for detecting a drug metabolite for isoniazid drugs is disclosed, it should be understood different biomarkers or metabolites may be detected using the test apparatuses disclosed herein. For example, B-vitamins such as niacin or nicotinic acid (from cigarette smoking) and the like may be detected using at least some of or similar reagents and color change reagents as disclosed with respect to isoniazid drug biomarkers herein. In some embodiments, different reagents and color change reagents may be used to identify different biomarkers in a similar manner as those disclosed above with respect to FIG. 3.

Returning to FIGS. 1 and 2, the indicator strip 120 (or 320) and control strip 110 (or 320) may be retained in test packaging to prevent the reactive gas from leaking into the environment outside of the test packaging. For example, the test packaging (e.g., casing) at least partially seals one or more portions of the control strip 110 and indicator strip 120 from the external environment. The test packaging 130 may include the base 150 and cover 140.

The control strip 110 and the indicator strip 120 may be disposed on the base 150. The cover 140 may be disposed on the base 150 to at least partially seal one or more portions of the control strip 110 and indicator strip 120 from an external environment. The base 150 and the cover 140 are sized, shaped, and composed to seal with each other on one or more portions thereof, such as in a medial region and terminal region 107. For example, the indicator strip 120 and control strip 110 may be sealed in at least at a terminal region 107 of the test apparatus 100, to prevent gases produced from the reagents thereon from leaking into the external environment.

The indicator strip 120 and control strip 110 may be exposed to the external environment at the initial region 105 of the test apparatus 100 (e.g., portion where the biological sample is applied) to allow the biological sample to be applied to the control strip 110 and the indicator strip 120. For example, the cover 140 may include an access port 142 to allow a user to apply the biological sample to the indicator strip 120 and control strip 110 in the initial region 105. Accordingly, the test apparatus 100 can be used without risking leak of chemicals therefrom, such as the reactive gas.

The base 150 includes a control position 152 for the control strip 110 and an indicator position 154 for the indicator strip 120. The control position 152 is spaced from the indicator position 154 by a distance, such as any of the distances between the indicator strip 120 and the control strip 110 disclosed herein. As shown, in some examples, the control position 152 may be substantially parallel to the indicator position 154. The control position 152 and indicator position 154 may be equipped for retaining the control strip 110 and indicator strip 120 thereon, respectively, such as an adhesive or mechanical retention structures (e.g., clips, channels, compression between the base 150 and cover 140, or combinations of any of the foregoing).

The base 150 or portions thereof may be made of a polymer that does not react with any of the biological sample, the plurality of reagents, the biomarker, the color change reagents, or any other contents of the test apparatus 100. The base 150 or portions thereof may be constructed of polyethylene terephthalate, high density polyethylene, polyvinyl chloride, polycarbonate, polytetrafluoroethylene, polyether ether ketone, polyimides, an acrylic, or the like. The base 150 or portions thereof may be constructed of a hydrophobic material. The base 150 may include one or more portions that are paper such as in a polymer coated paper or cardboard.

The base 150 may be elongated, such as sized to be longer than the indicator strip 120 and the control strip 110. The base 150 may have a width sufficient to retain the indicator strip 120 and the control strip 110 thereon and spaced from each other by the distance (e.g., 1 mm to 2 cm). The base 150 may include sidewalls extending upward around at least a portion of the periphery thereof, such as to create a recessed area therein. It should be understood that test packaging 130 is just one embodiment for packaging for the control and indicator strips disclosed herein and there are alternative embodiments of test packaging with similar functionality to the test packaging 130 to allow the reactions disclosed herein to take place on the control and indicator strips and prevent leakage of reagents and products therefrom.

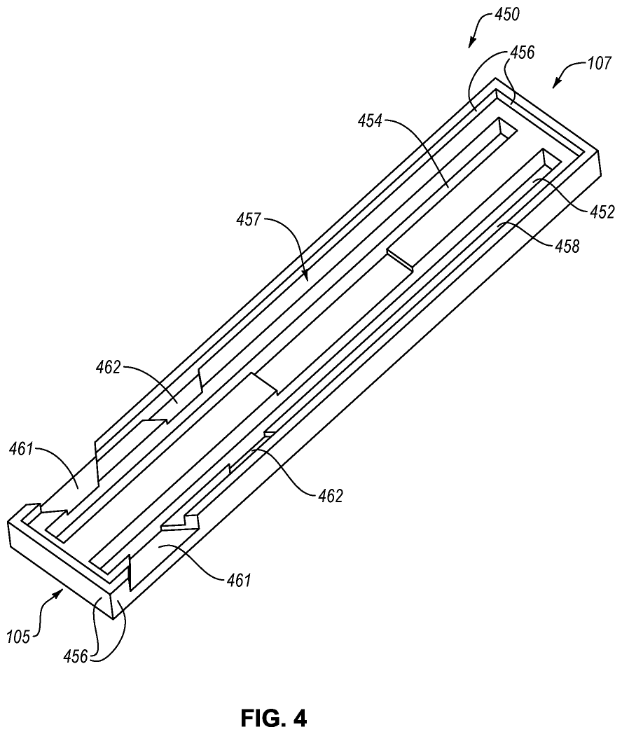

In some examples, the base 150 may include one or more structures to prevent the liquid sample from causing the solid and liquid contents of the indicator strip 120 and the control strip 110 to contaminate each other. FIG. 4 is an isometric view of a base 450, according to an embodiment. The base 450 may be similar or identical to the base 150 in one or more aspects, such as material composition, dimension, or the like. For example, the base 450 includes a body at least partially formed from a material that is not reactive with any of the biological sample, the plurality of reagents, the biomarker, the color change reagents, or any other contents of the test.

The base 450 includes a control position 452 for the control strip and an indicator position 454 for the indicator strip. The control position 452 and the indicator position 454 may be similar or identical to the control position 152 and the indicator position 154 in one or more aspects, respectively. For example, the control position 452 is spaced from the indicator position 454 by a distance, such as any of the distances between the indicator strip and the control strip disclosed herein. As shown, the control position 452 may be substantially parallel to the indicator position 454. The control position 452 and indicator position 454 may be equipped for retaining the control strip and indicator strip thereon, respectively. For example, the control position 452 may include a control strip channel formed in a floor 458 of the base 450 and the indicator position 454 may include an indicator strip channel formed in the floor 458 of the base 450. The channels may be a recessed area in the floor 458 of the base in size and shape of the indicator strip and control strip. The depth of the channels may be equal to or greater than the thickness of the indicator strip or control strip, respectively. The control strip may be disposed in the control strip channel and the indicator strip may be disposed in the indicator strip channel. In such examples, the biological sample (e.g., urine) and reagents may be retained within the respective channel(s), excluding the reactive gas. In some examples, the material on the floor 458 between the control strip channel and the indicator strip channel may be removed in some positions along the channels so that the reactive gas produced in the control strip can contact the indicator strip. For example, the material between the control channel and indicator channel may be removed in a medial region of the base 450 to allow the reactive gas produced on the control strip to migrate to the indicator strip in the indicator strip channel.

The base 450 may include one or more sidewalls 456 extending upward around one or more portions of the periphery of the base 450. The one or more sidewalls 456 may at least partially define a recessed area 457 within the base 450. The recessed area 457 may be at least partially defined by the floor 458 of the base 450. The interior of the one or more sidewalls 456 may be sized and shaped to engage with (e.g., seal against) the cover 140 (FIGS. 1, 2, and 5), such as at the peripheral edge of the cover 140. The top of the one or more sidewalls 456 may be sized and shaped to engage with (e.g., seal against) the cover 140 (FIGS. 1, 2, and 5), such as at a peripheral edge the bottom surface of the cover 140.

The base 450 may include one or more structures for ensuring that excess biological sample (e.g., liquid) does not cause the test to fail due to contamination of the strips cause by flooding. For example, the base 450 may include one or more gutters for retaining excess biological sample therein. The gutters may include one or more of an initial gutter 461 or a sealed gutter 462. The gutters may be recesses formed in the floor 458. The initial gutters 461 may be located in the initial region 105, so that excess biological sample in the initial region can be moved away from the channels. In some embodiments, the initial gutters 461 may be connected to or otherwise in fluid communication with the control channel and indicator channel. At least some of the recesses of the gutters may be separated from the control channel and indicator channel. For example, the initial gutters 461 may be separated from the control channel and indicator channel. In such examples, the excess biological sample may be isolated from the control channel and indicator channel by draining into the initial gutters 461. In some examples, the one or more sidewalls 456 may be omitted where the initial gutters extend outward. Accordingly, the excess biological samples may drain out of the test via the initial gutters and gaps in the sidewalls 456.

The sealed gutters 462 may be located in a portion of the base 450 that is sealed from the external environment when a cover is applied thereto. The sealed gutters 462 may be located in a medial region of the base between the initial region 105 and the terminal region 107. At least some of the recesses of the gutters may be in fluid communication with the control channel and indicator channel. For example, the sealed gutters 462 may be connected to the channels such that excess biological sample in the control channel and indicator channel or control strip and indicator strip is routed out of the control channel and indicator channel or control strip and indicator strip. The material between the control channel and indicator channel may be removed after the sealed gutters 462 (e.g., longitudinally closer to the terminal region 107) to ensure that no excess liquid is in the control strip and indicator strip when the material therebetween is not present. For example, the material between the control channel and indicator channel may be removed in a medial portion of the base 450 between the initial region 105 and the terminal region 107. Such configurations prevent liquid and materials therein of one strip from contaminating another strip, while allowing the reactive gas to migrate from the control strip to the indicator strip.

Returning to FIGS. 1 and 2, the cover 140 is sized and shaped to seal around at least a portion of a periphery of the base 150 (or 450) to at least partially seal one or more portions of the control strip 110 and indicator strip 120 in a sealed region therebetween. The one or more portions of the control strip 110 and the indicator strip and contents thereof (e.g., reagents, color change reagent, biomarker) in the sealed region are at least partially sealed from the external environment, such as to at least partially prevent the reactive gas from leaking out of the sealed region. The cover 140 may be sealed to the base 150 by an adhesive, weld, compression fit, coating, or the like on one or more portions thereof.

The cover 140 may be constructed of one or more of a polymer paper, paperboard, a polymer, a glass, or the like. For example, the cover 140 may be formed from any of the same polymers as the base 150. In some examples, the cover 140 may include a portion of one material and at least a second portion including at least a second material. For example, the cover 140 may include a polymer coated paper or paperboard. In some examples, the cover 140 may include a clear polymer (e.g., acrylic, polycarbonate, etc.) or a glass (e.g., quartz glass, borosilicate glass, etc.) window 144.

The cover includes an access port 142 in the initial region 105 thereof and a window 144 in the terminal region 107. The cover 140 may include one or more of an orientation marker 146 or a test identifier 148 on an outward facing portion thereof. The cover 140 may include a control strip identifier 147 and an indicator strip identifier 149 on the outward facing portion thereof. The access port 142 may be configured as a well, hole, gap, or other discontinuity in the cover 140. The access port 142 may be located in the initial region 105, such as over the initial ends of the control strip 110 and the indicator strip 120 to allow the same to be exposed to the external environment, such as for biological sample application thereto.

In some embodiments (not shown), the cover 140 may optionally include a flap or other sealing structure for covering the access port 142 after application of biological sample to the test apparatus 100.

The cover 140 includes the window 144 in the terminal region 107. The window may be made of a clear material such as a clear polymer (e.g., acrylic, polycarbonate, etc.) or a glass (e.g., quartz glass, borosilicate glass, etc.). The window may be the same material as the rest of the cover 140 or a may be a different material. For example, the main body of the cover 140 may be made of a first polymer and the window 144 may be a second clear polymer or glass. The window 144 is positioned over the terminal regions of the control strip 110 and the indicator strip 120. Accordingly, the control strip 110 and the indicator strip 120 may be viewed through the window to interpret the results of the test apparatus 100 (e.g., confirm presence of colored compound or additional colored compound). In some embodiments, the window 144 may include a window over the control strip 110 and another window over the indicator strip 120.

The cover 140 may include the orientation marker 146 to allow machine vision software to identify the orientation of the test apparatus 100 in a photograph. The orientation marker 146 may be printed on, applied to, or otherwise be visible from the outward facing surface of the cover 140. For example, the orientation marker 146 may printed on the cover 140 in ink, may be applied as a sticker, or may be painted on the cover 140. The orientation marker 146 may include a pattern of shapes that identify the relative orientation of the test apparatus 100 in three dimensional space with respect to the initial region 105 and terminal region 107. Accordingly, the orientation maker allows the test apparatus 100 to be accurately read and interpreted automatically by a machine vision system stored in a computing device, to confirm adherence to a pharmaceutical treatment regimen. The orientation marker 146 may include a plurality of shapes disposed in a pattern than is unique in each of the x and y axes.

The cover 140 may include the test identifier 148 on or otherwise visible from the outward facing surface of the cover 140. The test identifier 148 may include one or more of a bar code, an alphanumeric identification code, a QR code, or the like. The test identifier 148 may be unique to one or more of the individual test apparatus 100, the patient, a medical care provider (e.g., doctor), the manufacturer, or the like. Accordingly, the test identifier 148 may be used to confirm that a test apparatus 100 in a photograph is a new test apparatus 100, or that the photograph of a used test apparatus 100 is not a copy of a photograph of a previously used and submitted test apparatus 100. Further, the test identifier 148 may also be used by a computing system to automatically direct the electronic copy of the photograph of a completed test apparatus 100 to the electronically stored medical records of the patient associated with the test identifier. Such automatic routing may allow automatic tracking and confirmation of compliance or non-compliance with a prescribed pharmaceutical treatment regimen as well as automatic instructions to the patient for further treatment.

The cover 140 may include control strip identifier 147 and indicator strip identifier 149. The control strip identifier 147 and indicator strip identifier 149 may be located on the cover 140 in proximity to the control strip 110 and indicator strip 120 therebeneath. The control strip identifier 147 and indicator strip identifier 149 may include a textual indication of the strip therebeneath, such as the word “control” or “indicator” or may include the initial “C” for control or “I” for indicator.

Cover 140 and the base 150 may include one or more retention components, such as tabs, recesses, a size and shape to form a compression fit therebetween, or the like. Accordingly, the cover 140 and base 150 may be assembled and maintain a seal around at least a portion of the control strip and indicator strip therebetween to prevent reactive gas from leaking into the external environment outside of the sealed portion of the test apparatus 100.

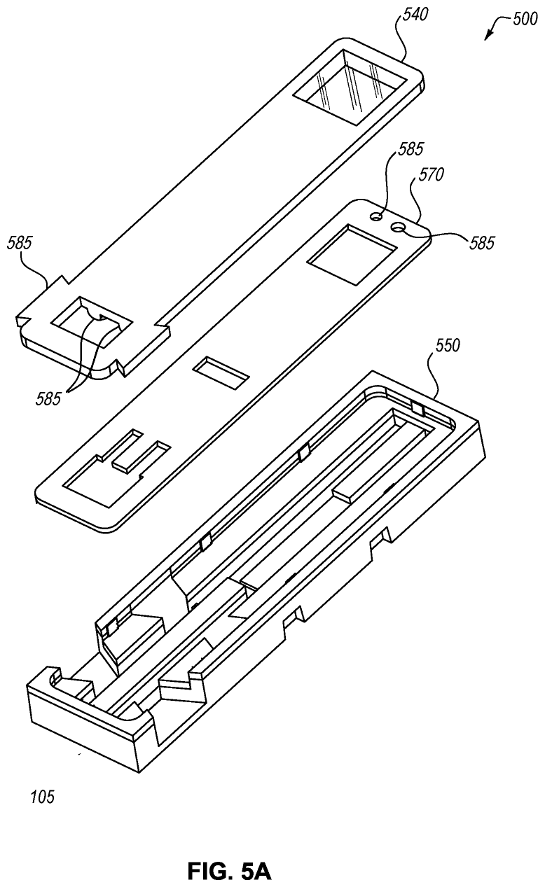

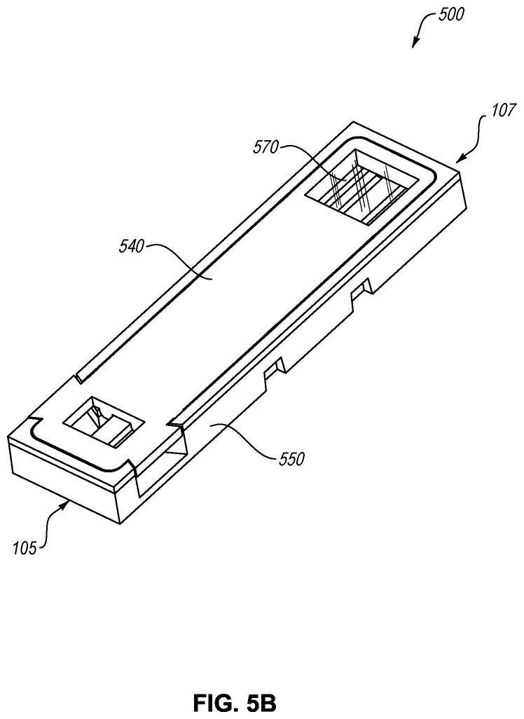

Further sealing structures may be provided by the test packaging. For example, a gasket may be present between the cover 140 and the base 150. FIG. 5A is an exploded isometric view of a test apparatus 500 according to an embodiment. FIG. 5B is an isometric view of the test apparatus 500, according to an embodiment. The test apparatus 500 includes the cover 540, the base 550, and a gasket 570, collectively forming at least a portion of the test packaging. The cover 540 may be similar or identical to any of the covers disclosed herein, in one or more aspects. The base 550 may be similar or identical to any of the bases disclosed herein, in one or more aspects. The gasket 570 is disposed between the cover 540 and the base 550.

The gasket 570 is sized and shaped to form a seal between the cover 540 and the base 550 when the cover 540 and base 550 are assembled with the gasket therebetween. For example, the gasket 570 may be sized and shaped to fit at least partially within one or more portions of the base 550, such as in the recessed area therein. In some examples, the gasket 570 may be sized and shaped to fit on the perimeter of the base 550 or around the perimeter of the cover 540. The gasket 570 may be constructed of a polymer (e.g., elastomers, silicone), natural material (e.g., rubber), or any other material suitable for sealing surfaces to prevent leakage of fluid components.

One or more of the cover 540, the base 550, or the gasket 570 may include one or more alignment structures 585 thereon. The one or more alignment structures 585 may include any of protrusions, depressions, or holes that align with each other for ensuring proper alignment upon assembly. For example, the cover 540 may include one or more protrusions or tabs that align with one or more depressions or cut-outs in the base 550. The gasket 570 may include one or more holes or cut-outs therein that align with the protrusions or tabs on the cover 540 to align the gasket with both the base 550 and cover 540 upon assembly. Once assembled, the gasket 570 is aligned with the cover 540 and base 550 to provide a fluid tight seal therebetween. Accordingly, the seal created by the cover 540 and/or gasket 570 with the base 550 may allow biological sample fluid to flow from the initial region toward the terminal region via control and indicator strips, but seal the medial and terminal regions from leaking reactive gas or other reagents and reactants therefrom.

The test apparatus 500 may include any of the control strips and indicator strips disclosed herein.

The test apparatuses disclosed herein may be constructed of materials that are stable at temperatures between −5° C. and 50° C. and humid or dry environments alike. The tests apparatuses may be stable for at least 1 month, such as 1 month to 3 years, 9 months to a year, or 6 months to 2 years, without losing effectiveness or functionality. In some embodiments, the test apparatus may be packed with desiccant in a sealed bag, such as a Mylar bag or the like.

The tests apparatuses disclosed herein can be used to test for biomarkers such as drug metabolites in a patient's system, such as in urine. The tests apparatuses disclosed herein can be used to confirm that a patient is complying with (e.g. adhering to) a pharmaceutical treatment regimen. For example, the tests apparatuses disclosed herein can be used to treat tuberculosis by determining if a patient is complying with a pharmaceutical treatment regimen for TB by determining if the patient has biomarkers for a TB drug such as INH in there system, without ever being in the same location as the patient.

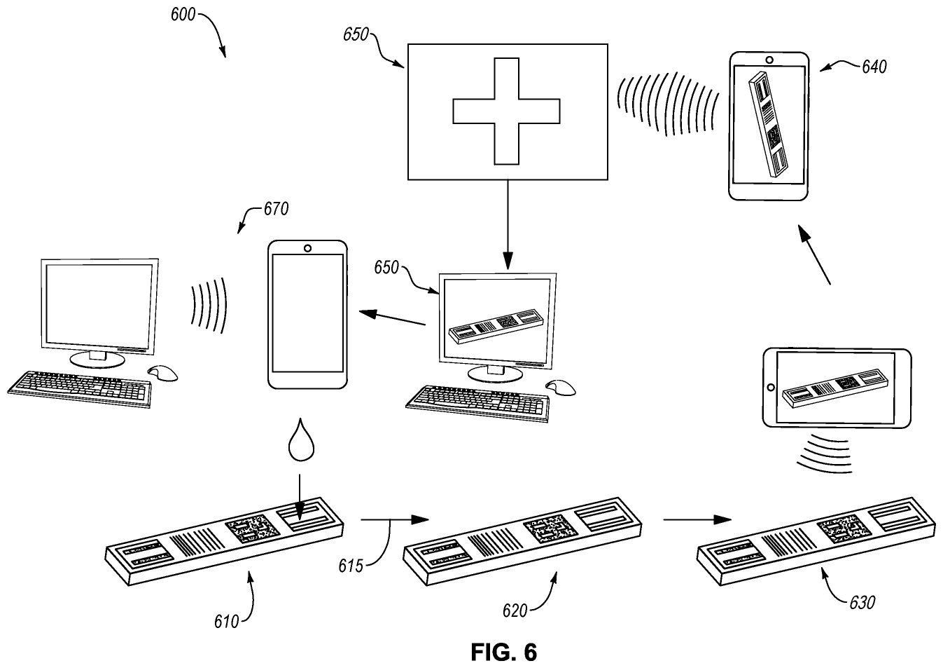

FIG. 6 is a flow diagram of a method 600 for testing for a biomarker and treating a patient for a condition, according to an embodiment. The method 600 includes block 610 of applying a biological sample onto a test apparatus for a biomarker; the block 615 of allowing the biological sample to travel through the control strip and indicator strip to a terminal region thereof for a duration of time sufficient to allow the color change reagent to form the colored compound if the biomarker is present in the biological sample; and the block 620 of identifying a presence or absence of the colored compound on the indicator strip; the block 630 of taking a photograph of the test apparatus after allowing the biological sample to travel through the control strip and indicator strip; the block 640 of sending an electronic copy of the photograph to a medical provider via a wireless network; the block 650 of receiving an electronic copy of a photograph of a completed biomarker test via a wireless network; the block 660 of identifying a presence or absence of the colored compound on the indicator strip; and the block 670 of providing a treatment regimen responsive to the presence or absence of the colored compound on the indicator strip. In embodiments, one or more of the blocks 610-670 may be omitted, combined, or performed in a different order than presented herein. For example, a method for testing for a drug biomarker may include blocks 610-640 and a method for treating a condition (e.g., TB) may include blocks 650-670. In some embodiments, one or more of the blocks 610-670 may be split into multiple blocks. For example, the block 660 of identifying a presence or absence of the colored compound on the indicator strip may be split into a plurality of blocks to identify the presence or absence using a machine vision system.

As noted above the method 600 includes a method for testing for a drug biomarker in blocks 610-640. The block 610 of applying a biological sample onto a test apparatus for a biomarker may include utilizing any of the test apparatuses disclosed herein. The test apparatus may include any of the indicator strips, control strips, reagents, color change reagents, additional color changes reagents, biomarkers or biomarker analogues, or test packaging (e.g., bases and covers) disclosed herein. For example, the test apparatus may include a control strip including a plurality of (e.g., at least three) reagents formulated to make a reactive gas upon mixing, and an indicator strip spaced from the control strip, the indicator strip including a color change reagent formulated to form a colored compound upon reaction with a biomarker and the reactive gas.

In embodiments, the plurality of reagents on the control strip may include chloramine T, potassium thiocyanate, and citric acid. The color change reagent and addition color change reagents on the indicator strip and control strip, respectively, may include barbituric acid. The biomarker on the control strip and potentially in the biological sample may include an isoniazid drug or metabolite thereof, such as INH or INA.

Applying a biological sample onto a test apparatus for a biomarker includes applying (e.g., dropping) a liquid biological sample such as urine onto the control strip and indicator strip at an initial region thereof, such as via an access port on the cover of the test apparatus. The biological sample may be applied to the initial regions of the control strip and indicator strip.

The block 615 of allowing the biological sample to travel through the control strip and indicator strip to a terminal region thereof for a duration of time sufficient to allow the color change reagent to form the colored compound if the biomarker is present in the biological sample may include placing the test apparatus on a flat surface. The duration may include at least 1 minute, such as 1 minute to 30 minutes, 3 minutes to 20 minutes, 3 minutes to 5 minutes, 5 minutes to 10 minutes, 10 minutes to 20 minutes, less than 20 minutes, less than 10 minutes, or less than 5 minutes. For example, allowing the biological sample to travel through the control strip and indicator strip may include includes waiting 3 to 5 minutes after applying the biological sample.

In some embodiments, the control strip may include an additional color change reagent and the biomarker or a biomarker analogue, where the additional color change reagent is formulated to form an additional colored compound on the control strip upon reaction with the biomarker or biomarker analogue. In such embodiments, the block 615 may include allowing the biological sample to travel through the control strip to a terminal region thereof for a duration of time sufficient to allow the additional color change reagent to form the additional colored compound. The duration of time may include any of those durations disclosed above, such as 3 to 5 minutes.

The block 620 of identifying a presence or absence of the colored compound on the indicator strip may include visually identifying a change of the color of the indicator strip, such as at the terminal region thereof. For example, identifying a presence or absence of the colored compound on the indicator strip may include visually identifying a blue-purple color on the indicator strip. Identifying a presence or absence of the colored compound on the indicator strip may be carried out by the user of the test apparatus, such as a patient, care provider, or the like.

The block 630 of taking a photograph of the test apparatus after allowing the biological sample to travel through the control strip and indicator strip may include taking a photograph of the test apparatus. The photograph may be a digital or electronic photograph capable of being electronically transmitted, such as wirelessly. Taking the photograph may be done with a camera on a cellular telephone (e.g., smart phone). Taking the photograph may include taking a photograph of the top side of the test apparatus effective to capture at least the terminal region of the control strip and indicator strip in the photograph. In embodiments, one or more of the window, orientation marker, test identifier, or the access port are included in the photograph. In such examples, the orientation marker may be visible in the photograph to allow a machine vision program to determine the three dimensional orientation of the test apparatus; the test identifier may be visible to allow the machine vision program to associate the test apparatus with a user, medical professional, or the valid test; and the window may be visible to allow the terminal regions of the control strip and indicator strip to be viewed in the photograph.

Block 640 of sending an electronic copy of the photograph to a medical provider via a wireless network may include sending the photograph to the medical care provider directly, such as to a doctor, a nurse, a medical clinic, or to application software (“App”) accessible by the medical care provider. For example, sending an electronic copy of the photograph to a medical provider via a wireless network may include sending the electronic copy of the photograph by text, electronic mail, an App, or peer to peer network. The App may include a web-based interface to allow a care provider to track individual patients over time. The App may include machine readable and executable programs to provide functions including education on the disease and its treatment, self-reporting of medication administration and side-effects, image capture of the drug metabolite test, progress feedback, reminders, and an anonymous group chat to answer questions and help reduce stigma and lack of understanding about a condition and its treatment. Such functions may be used, at least in part, to determine and provide a treatment regimen by the care provider. When the patient uses the App, the App can be capture and store data such as the electronic copy or copies of the photograph(s), then transmitted to the care provider when an internet or cellular connection has been made with the patient's cellular phone.

In some embodiments, the control strip may include an additional color change reagent and the biomarker or a biomarker analogue, wherein the additional color change reagent is formulated to form an additional colored compound on the control strip upon reaction with the biomarker or biomarker analogue. In such examples, allowing the biological sample to travel through the control strip and indicator strip includes allowing the biological sample to travel through the control strip to a terminal region thereof for a duration of time sufficient to allow the additional color change reagent to form the additional colored compound and identifying the presence of the additional colored compound on the control strip. In such examples, the color change reagent and the additional color change reagent may be the same chemical species, such as barbituric acid, which provides a blue-purple color. In embodiments where the control strip includes the additional color change reagent and the biomarker or a biomarker analogue, the method 600 may include identifying the presence of the additional colored compound on the control strip. Identifying the presence of the additional colored compound on the control strip may be similar or identical to identifying the presence of the presence or absence of the colored compound on the indicator strip, in one or more aspects, such as via a machine vision system.

The applying, allowing, identifying, taking, and sending steps may be performed periodically (e.g., for weeks or months) to demonstrate compliance or long term compliance with a pharmaceutical treatment regimen.

The medical care provider may view the electronic copy of the photograph to visually confirm that the patient is complying or not complying with the pharmaceutical treatment regimen. The medical care provider may then provide a further treatment regimen to the patient at least partially based on the results of the test(s) in the photograph(s).

The method 600 includes the method for treating a condition (e.g., TB) at blocks 650-670. Block 650 of receiving an electronic copy of a photograph of a completed biomarker test via a wireless network may include receiving the electronic copy of any of the test apparatuses disclosed herein, that have had a biological sample applied thereto. For example, the completed biomarker test may include a test apparatus having a control strip including at least three reagents formulated to make a reactive gas upon mixing, and an indicator strip spaced from the control strip, the indicator strip including a color change reagent formulated to form a colored compound upon reaction with a biomarker and the reactive gas. Receiving an electronic copy of a photograph of a completed biomarker test via a wireless network may include receiving the electronic copy of the photograph of a used test apparatus at a computing system (e.g., server(s) or computer(s)) of a care provider or service provider thereto.

Block 660 of identifying a presence or absence of the colored compound on the indicator strip may include visually identifying the absence or presence of the colored compound on the indicator strip. Identifying a presence or absence of the colored compound on the indicator strip may be carried out by a machine vision system, such as via machine vision system software program (e.g., machine readable and executable program) stored in a computing system or a separate computing system operably connected thereto (e.g., servers or computers). The machine vision software program may include instructions to locate, identify, and view the terminal region of the indicator strip in the electronic copy of the photograph. Identifying a presence or absence of the colored compound on the indicator strip may include determining a location of the terminal region of the indicator strip by determining an orientation of the test apparatus using the orientation marker. For example, the program may include instructions to view the orientation marker on the cover the test packaging to identify the orientation of the test apparatus. Based on the determined orientation of the test apparatus the program may include instructions to identify the projected location of the terminal region of the indicator strip. The program may include instructions to search the pixels in the copy of the electronic image in the projected locate on the terminal region of the indicator strip for a selected color, such as the expected color of the colored compound (e.g., blue-purple color).

Identifying the presence of the presence or absence of the colored compound on the indicator strip may include finding identifying the test identifier. The test identifier may be utilized for one or more of confirming the test is a valid test, correlating the results to the patient or a medical provider, or correlating the results to the data in the image, such as automatically by machine readable and executable instructions stored in the computing system.

In some embodiments, the control strip may include an additional color change reagent and the biomarker or a biomarker analogue, where the additional color change reagent is formulated to form an additional colored compound on the control strip upon reaction with the biomarker or biomarker analogue. In such embodiments, the block 660 may include identifying the presence of the additional colored compound on the control strip. Identifying the presence of the additional colored compound on the control strip may be similar or identical to identifying the presence of the presence or absence of the colored compound on the indicator strip, in one or more aspects, such as via a machine vision system. A medical care provider or agent thereof may visually identify the presence or absence of the colored compound and additional colored compound on the terminal regions of the indicator strip and control strip.

The block 670 of providing a treatment regimen responsive to the presence or absence of the colored compound on the indicator strip may sending instructions to the patient at a source of the electronic copy of the photograph to maintain or alter the pharmaceutical treatment regimen based on the presence or absence of the colored compound in the electronic copy of the photograph. For example, providing a treatment regimen responsive to the presence or absence of the colored compound on the indicator strip may include automatically sending instructions to a source of the electronic copy of the photograph to maintain or discontinue using an isoniazid drug for TB treatment. Providing a treatment regimen responsive to the presence or absence of the colored compound on the indicator strip may include providing the treatment regimen based on long term compliance with the pharmaceutical regimen. For example, the medical care provider may send instructions to the patient to terminate the pharmaceutical treatment because they have complied with the pharmaceutical treatment for a satisfactory duration of time.

The method 600 may include repeating the receiving and identifying steps to determine compliance with a pharmaceutical regimen long term.

Given that over 95% of the global population now lives in areas that are covered by a mobile-cellular network and that smartphone ownership is rapidly rising, mobile tools for treatment support can improve delivery of evidence-based interventions and patients' engagement in care. Accordingly, the apparatuses, systems, and methods disclosed herein provide a remote interface for patient engagement to monitor compliance with pharmaceutical treatment regimens. The apparatuses, systems, and methods herein provide for visual indication of biomarkers in biological samples without the risk of false positives based on the separation of the control and test strips and the use of the reactive gas from the control strip for the formation of the colored compound on the indicator strip. Further, the apparatuses herein may be photographed by users and sent to care providers, such as via an App, for confirmation of adherence to a pharmaceutical treatment regimen.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A test apparatus for detecting a drug metabolite, the test apparatus comprising:
a control strip including at least three reagents formulated to make a reactive gas upon mixing; and
an indicator strip spaced from the control strip by a distance, the indicator strip including a color change reagent formulated to form a colored compound upon reaction with a biomarker and the reactive gas.

2. The test apparatus of claim 1 wherein the at least three reagents include sodium chloro(4-methylbenzene-1-sulfonyl)azanide, 2-hydroxypropane-1,2,3-tricarboxylic acid, and potassium thiocyanate.

3. The test apparatus of claim 1 wherein the control strip includes an additional color change reagent formulated to form an additional colored compound upon reaction with the biomarker or a biomarker analogue.

4. The test apparatus of claim 1, further comprising a test packaging at least partially sealing one or more portions of the control strip and indicator strip from an external environment.

5. The test apparatus of claim 4 wherein:
the test packaging includes a base having a control strip channel formed therein and an indicator strip channel formed therein and spaced the distance from the control strip channel;
the control strip is disposed in the control strip channel; and
the indicator strip is disposed in the indicator strip channel.

6. The test apparatus of claim 5 wherein the test packaging includes a cover sized and shaped to seal around at least a portion of a periphery of the base to at least partially seal one or more portions of the control strip and indicator strip therebetween and the cover includes an access port in an initial region thereof and a window in a terminal region thereof.

7. The test apparatus of claim 6 wherein the cover includes one or more of an orientation marker or a test identifier.

8. The test apparatus of claim 6 wherein the indicator strip is substantially parallel to the control strip.

9. The test apparatus of claim 1 wherein the biomarker includes one or more of 4-pyridinecarboxylic acid hydrazide, 4-pyridinecarboxylic acid, or derivatives thereof.

10. A method for testing for a drug metabolite, the method comprising:
applying a biological sample onto a test apparatus for a biomarker, the test apparatus including:
a control strip including at least three reagents formulated to make a reactive gas upon mixing, and
an indicator strip spaced from the control strip, the indicator strip including a color change reagent formulated to form a colored compound upon reaction with a biomarker and the reactive gas,
allowing the biological sample to travel through the control strip and indicator strip to a terminal region thereof for a duration of time sufficient to allow the color change reagent to form the colored compound if the biomarker is present in the biological sample; and
identifying a presence or absence of the colored compound on the indicator strip.

11. The method of claim 10 wherein applying a biological sample onto a test for a biomarker includes applying urine onto the control strip and indicator strip at an initial region thereof.

12. The method of claim 10 wherein allowing the biological sample to travel through the control strip and indicator strip includes waiting 3 to 5 minutes after applying the biological sample.

13. The method of claim 10, further comprising:
taking a photograph of the test apparatus after allowing the biological sample to travel through the control strip and indicator strip; and
sending an electronic copy of the photograph to a medical provider via a wireless network.

14. The method of claim 13 wherein:
taking a photograph of the test apparatus includes taking the photograph with a cellular phone; and
sending an electronic copy of the photograph to a medical provider via a wireless network includes sending the electronic copy of the photograph by text, electronic mail, or peer to peer network.

15. The method of claim 14, further comprising repeating the applying, allowing, identifying, taking, and sending steps periodically to demonstrate compliance with a pharmaceutical regimen.

16. A method for treating tuberculosis, the method comprising:
receiving an electronic copy of a photograph of a completed biomarker test via a wireless network, the completed biomarker test including a test apparatus having,
a control strip including at least three reagents formulated to make a reactive gas upon mixing, and
an indicator strip spaced from the control strip, the indicator strip including a color change reagent formulated to form a colored compound upon reaction with a biomarker and the reactive gas,
identifying a presence or absence of the colored compound on the indicator strip; and
providing a treatment regimen responsive to the presence or absence of the colored compound on the indicator strip.

17. The method of claim 16 wherein the test apparatus includes test packaging at least partially sealing one or more portions of the control strip and indicator strip from an external environment, the test packaging including:
a base for retaining the control strip and indicator strip thereon; and
a cover sized and shaped to seal around at least a portion of a periphery of the base to at least partially seal one or more portions of the control strip and indicator strip therebetween and the cover includes,
an outward facing surface having an orientation marker and a test identifier thereon,
an access port in an initial region of the cover, and
a window in a terminal region of the cover.

18. The method of claim 17, wherein identifying a presence or absence of the colored compound on the indicator strip includes determining a location of the indicator strip by determining an orientation of the test apparatus using the orientation marker.

19. The method of claim 17, wherein providing a treatment regimen responsive to the presence or absence of the colored compound on the indicator strip includes automatically sending instructions to a source of the electronic copy of the photograph to maintain or discontinue using an isoniazid drug.

20. The method of claim 17, further comprising repeating the receiving and identifying steps to determine compliance with a pharmaceutical regimen.
